(19) United States
(12) Patent Application Publication
(10) Pub. No.: US 2018/0043182 A1
(43) Pub. Date: Feb. 15, 2018

(54) SYSTEMS AND METHODS FOR AUTOMATED RADIATION TREATMENT PLANNING WITH DECISION SUPPORT

(71) Applicant: Duke University, Durham, NC (US)

(72) Inventors: Qingrong Jackie Wu, Durham, NC (US); Yaorong Ge, Durham, NC (US); Fang-Fang Yin, Durham, NC (US); Lulin Yuan, Durham, NC (US); Yang Sheng, Durham, NC (US); Taoran Li, Durham, NC (US); Jianfei Liu, Durham, NC (US)

(21) Appl. No.: 15/555,488

(22) PCT Filed: Mar. 7, 2016
(86) PCT No.: PCT/US16/21271

§ 371 Date: Sep. 1, 2017

Related U.S. Application Data
(60) Provisional application No. 62/129,125, filed on Mar. 6, 2015.

(51) Int. Cl. A61N 5/10; G06F 19/00

(52) U.S. Cl. CPC A61N 5/103; A61N 5/1039; A61N 5/1031; G06F 19/345; G06F 19/3431; G06F 19/3437; G06F 19/325; A61N 2005/1041; A61N 2005/1074; G06F 19/3406

(57) ABSTRACT

Systems and methods for automated radiation treatment planning with decision support are disclosed. According to an aspect, a method includes receiving data based on patient information and geometric characterization of one or more organs at risk and a cancer target of a patient. The method also includes determining the appropriate models and model settings for the given patient case. Further, the method includes generating automatically one or more radiation treatment plans using the proper models learned from a plurality of radiation treatment plans of prior patient cases based on certain relationships, including one of a match or similarity, between the patient information and geometric characterization of the patient and the other patients. The method also includes presenting the determined one or more radiation treatment plans via a user interface.

### CROSS REFERENCE TO RELATED APPLICATIONS

This application claims the benefit of and priority to U.S. Provisional Patent Application No. 62/129,125, filed Mar. 6, 2015 and titled SYSTEMS AND METHODS FOR AUTOMATED RADIATION TREATMENT PLANNING WITH DECISION SUPPORT; the disclosure of which is incorporated herein by reference in its entirety.

This application is related to PCT International Application Number ______, filed simultaneously herewith and titled SYSTEMS AND METHODS FOR EFFICIENT AND AUTOMATIC DETERMINATION OF RADIATION BEAM CONFIGURATIONS FOR PATIENT-SPECIFIC RADIATION THERAPY PLANNING.

This application is related to U.S. patent application Ser. No. 14/893,055, titled SYSTEMS AND METHODS FOR SPECIFYING TREATMENT CRITERIA AND TREATMENT PARAMETERS FOR PATIENT SPECIFIC RADIATION THERAPY PLANNING and filed Nov. 21, 2015.

### FEDERALLY SPONSORED RESEARCH OR DEVELOPMENT

This invention was made partially with government support under grant number R21CA161389 awarded by the National Institutes of Health (NIH). The government has certain rights to this invention.

### TECHNICAL FIELD

The presently disclosed subject matter relates to radiation therapy. Particularly, the presently disclosed subject matter relates to systems and methods for automated radiation treatment planning with decision support.

### BACKGROUND

Radiation therapy, or radiotherapy, is the medical use of ionizing radiation to control malignant cells. Radiation treatment planning involves complex decision making in specifying optimal treatment criteria and treatment parameters that take into account all aspects of patient conditions and treatment constraints and also in utilizing the most appropriate optimization algorithms and parameters to reach an optimal treatment plan. It is desired that the plan achieves maximal tumor control while minimizing normal tissue damage. Decision support is needed for treatment criteria, treatment parameters, and often the trade-offs and interplays between the criteria and parameters and among the various parameters. Once the parameters that optimally meet the treatment are determined, a high quality treatment plan is automatically generated that leads to high quality radiation treatment for the specific patient.

Current practice relies on personal experience and loosely defined guidelines. While there are decision support tools for selecting treatment options, such as selecting surgery, chemotherapy, or radiation therapy, there is a desire to provide systems and techniques for radiation therapy decision making and radiation therapy treatment planning.

### BRIEF SUMMARY

Disclosed herein are systems and methods for automated radiation treatment planning with decision support. The present disclosure provides decision support systems and methods for physicians, planners, and other healthcare practitioners to specify treatment criteria and parameters. Systems and methods disclosed herein can automatically generate high quality plans once the decisions are made and may be used by healthcare practitioners to support the evaluation and selection of alternative plans based on various trade-off scenarios. Systems and methods disclosed herein can incorporate available evidence, experience, and knowledge of radiation therapy as disclosed herein.

According to an aspect, a method includes receiving data based on patient information and geometric characterization of one or more organs at risk and a cancer target of a patient. The method also includes determining the appropriate models and model settings for the given patient case. Further, the method includes generating automatically one or more radiation treatment plans using the proper models learned from a plurality of radiation treatment plans of prior patient cases based on certain relationships, including one of a match or similarity, between the patient information and geometric characterization of the patient and the other patients. The method also includes presenting the determined one or more radiation treatment plans via a user interface.

### BRIEF DESCRIPTION OF THE SEVERAL VIEWS OF THE DRAWINGS

The foregoing aspects and other features of the present subject matter are explained in the following description, taken in connection with the accompanying drawings, wherein:

FIGS. 1A-1D are images depicting the effects of tumor enclosure on dose distributions;

FIGS. 2A-2C are images showing the extraction of dose sub-images and planning target volume (PTV) contours to build AOFM and ASM;

FIGS. 3A-3E, which are images depicting five types of spatial relationships between spinal cords and PTVs, where dose images are overlaid on the CT images;

FIGS. 4A-4D are images depicting an example optical flow computation;

FIGS. 5A and 5B are images showing instances of AOFM and ASM, respectively;

FIGS. 6A-6C are images depicting a process of dose prediction;

FIGS. 7A-7D are graphs showing experimental DVH results on L-spine (FIG. 7A), C-spine (FIG. 7B), and T-spine (FIGS. 7C and 7D) SBRT plans in the testing dataset;

FIG. 8 are images depicting a comparison of the predicted dose (top row) and clinical dose (bottom row);

FIG. 9 is a flowchart of an example method for automated radiation treatment with decision support in accordance with embodiments of the present disclosure;

FIG. 10 is an image showing beam angle efficiency map (green contour) plotted on anatomy;

FIG. 11 depicts graphs of MCO engines for the Pareto front (PS) search; and

FIG. 12 illustrates a graph of how each MCO engine searches for the Pareto optimal plans.

### DETAILED DESCRIPTION

For the purposes of promoting an understanding of the principles of the present disclosure, reference will now be made to various embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

In accordance with embodiments, the presently disclosed subject matter provides an efficient response method by utilizing patient-specific anatomy and tumor geometry information and a beam bouquet atlas.

In accordance with embodiments, the presently disclose subject matter provides support for healthcare practitioners to make plan decisions. Instead of deciding dose parameters in target and organs at risk (OARs), healthcare practitioners can use systems and methods disclosed herein to decide amongst one or more final treatment plans that are patient specific, best achievable, and can take into account various patient conditions, treatment goals, and clinical tradeoffs. These systems and methods can benefit from various models for predicting best achievable dose parameters. The present disclosed subject matter can be used to automate all or nearly all of the process of generating treatment plans. Systems and methods disclosed herein can automatically determine and train a set of models that are optimal for a given database of prior cases, in automatically selecting one or more correct models to use for a given patient, in automatically improving the models given new data and/or evidence and in automatic generation of a set of best achievable plans on a Pareto surface that can allow healthcare practitioners to select a final plan that is clinically optimal.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

As referred to herein, the term “computing device” should be broadly construed. It can include any type of device including hardware, software, firmware, the like, and combinations thereof. A computing device may include one or more processors and memory or other suitable non-transitory, computer readable storage medium having computer readable program code for implementing methods in accordance with embodiments of the present disclosure. A computing device may be, for example, retail equipment such as POS equipment. In another example, a computing device may be a server or other computer located within a retail environment and communicatively connected to other computing devices (e.g., POS equipment or computers) for managing accounting, purchase transactions, and other processes within the retail environment. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD). A computing device can also include any type of conventional computer, for example, a laptop computer or a tablet computer. A typical mobile computing device is a wireless data access-enabled device (e.g., an iPHONE® smart phone, a BLACKBERRY® smart phone, a NEXUS ONE™ smart phone, an iPAD® device, or the like) that is capable of sending and receiving data in a wireless manner using protocols like the Internet Protocol, or IP, and the wireless application protocol, or WAP. This allows users to access information via wireless devices, such as smart phones, mobile phones, pagers, two-way radios, communicators, and the like. Wireless data access is supported by many wireless networks, including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone that operates over GPRS (General Packet Radio Services), which is a data technology for GSM networks. In addition to a conventional voice communication, a given mobile device can communicate with another such device via many different types of message transfer techniques, including SMS (short message service), enhanced SMS (EMS), multi-media message (MMS), email WAP, paging, or other known or later-developed wireless data formats. Although many of the examples provided herein are implemented on smart phone, the examples may similarly be implemented on any suitable computing device, such as a computer. The system may be implemented in a cloud computing environment.

As referred to herein, the term “user interface” is generally a system by which users interact with a computing device. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The presently disclosed subject matter provides systems and methods for automatically generating patient-specific, best achievable IMRT plans based on available data and evidence and to provide decision support for healthcare practitioners to select a best plan that takes into account unique patient conditions, clinical treatment goals, and other considerations (e.g., patient preference).

Treatment planning knowledge can be accumulated and collected via personal training and experience and published clinical trial study data. The planning knowledge and experience can be loosely linked to plan quality and treatment outcome. In accordance with embodiments, systems and methods disclosed herein can automate the planning process based on discovering, describing, extracting, and integrating expert knowledge and experience from multiple comprehensive knowledge sources.

In accordance with embodiments, systems and methods disclosed herein can analyze and extract patient anatomy and cancer targets into mathematically parameterized patterns and use the patterns to classify patient plans to build an expert atlas. The expert plan atlas can subsequently be used to determine dose prescription and treatment planning for new patient cases.

In accordance with embodiments, systems and methods disclosed herein can analyze and characterize patient anatomy in terms of mathematically parameterized features and associate these features to patterns of radiation beam angles. It is important to assist healthcare practitioners through the automatic determine of beam angles and examples are described in a related application. A resulting model can be used to determine radiation beam angles which can be used as templates to initialize patient planning configurations, or can also be used to guide the further adjustment or fine tuning of these beam angles based on patient's unique anatomy and clinical radiation dose constraints.

In accordance with embodiments, systems and methods disclosed herein can analyze and extract patient anatomy and cancer targets into mathematically parameterized features and associate these features to the voxel-level dose to the critical structures (e.g., spinal cord).

In accordance with embodiments, systems and methods disclosed herein can extract, represent, and process published treatment planning knowledge using standardized concepts, ontologies, and other knowledge representations.

In accordance with embodiments, systems and methods disclosed herein can provide a decision support system based on techniques disclosed herein. The systems and methods can enable healthcare practitioners to prescribe best treatment dose options for the tumor and best protective dose to critical organs based on knowledge models and patient unique information.

It is noted that patient unique information may include some or all information that can potentially influence a healthcare practitioner's decision on prescribing dose to the planning target volume (PTV) and each of the OARs. Factors may include, but are not limited to, a patient's previous radiation treatment, prior treatment dose, location, and dose volume information of prior treatment to each of the OARs, patient's physiological conditions, such as organ function analysis, transplant condition, patient preference and treatment goals, and the like.

Knowledge models that may be used can allow for the prediction of dose, dose volume histogram of a patient, and the like. Further, systems and methods disclosed herein may utilize a case-based reasoning mechanism to allow a choice of different knowledge models based on one or more clinical conditions that are relevant to the patient.

Given patient specific information and knowledge model-based dose prediction for each OAR, a healthcare practitioner may use the trade-off dose model to modify the dose prediction for a specific OAR. The population based OAR toxicity data may also be included as a factor to assist a physician or other healthcare practitioner to make a complex trade-off decision. For any change in on OAR's dose prediction, a dose trade-off model can predict its impact by updating the IMRT plan with dose predictions for the PTV and other OARs. The process can continue until the physician finished the trade-off process. Such systems can support the decision making of the physician with integrated, multi-source knowledge, and patient unique condition with one platform.

In accordance with embodiments, systems and methods disclosed herein can automatically determine an optimal set of models based on available prior datasets (referred to as “dataset partitioning”). Systems and methods disclosed herein can automatically select one or more models for a given patient case (referred to as “case based reasoning”). Systems and methods disclosed herein can capture decision variations and handle exceptions (referred to as “case based reasoning”). Systems and methods disclosed herein can also incrementally and progressively learn models. Further, systems and methods disclosed herein can produce a set of alternative optimal plans using a local MCO approach. Systems and methods disclosed herein can also utilize dose predictions models and beam configuration determinations.

In accordance with embodiments, treatment planning knowledge can be accumulated and collected via personal training and experience and published clinical trial study data. The planning knowledge and experience may be linked to plan quality and treatment outcome. Systems and methods disclosed herein can automate the planning process and be based on discovering, describing, extracting, and integrating expert knowledge and experience from multiple comprehensive knowledge sources.

Accurate dose predication can be important in spinal stereotactic body radiation therapy (SBRT). It can enable radiation oncologists and planners to design high-quality treatment plans of maximally protecting spinal cords and effectively controlling surrounding tumors. Dose distributions at spinal cords are primarily affected by the shapes of adjacent PTV contours. In embodiments, such contour effects are estimated and dose distributions predicted by exploring active optical flow model (AOFM) and active shape model (ASM). We first collect a sequence of dose sub-images and PTV contours near spinal cords from any suitable number (e.g., fifteen) SBRT plans in the training dataset. The data collection is then classified into any suitable number (e.g., five groups) according to the PTV locations in relation to spinal cords. In each group, we randomly choose a dose sub-image as the reference and register all other sub-images to the reference using an optical flow method. AOFM is then constructed by importing optical flow vectors and dose values into the principal component analysis (PCA). Similarly, we build ASM by using PCA on PTV contour points. The correlation between ASM and AOFM is estimated via a multiple-stepwise regression model. When predicting dose distribution of a new case, the group is first determined based on the PTV contour. The prediction model of the selected group is used to estimate dose distributions by mapping the PTV contours from the ASM space to the AOFM space in terms of the correlation parameters. This method was validated on any suitable number (e.g., fifteen) SBRT plans in the testing dataset. Analysis of dose-volume histograms revealed that at the important 2%, 5% and 10% volume mark, the dose level of prediction and clinical plan were 11.7±1.7 Gy vs. 11.8±1.7 Gy (p=0.95), 10.9±1.7 Gy vs. 11.1±1.9 Gy (p=0.8295), and 10.2±1.6 Gy vs. 10.1±1.7 (p=0.9036). These results suggested that the AOFM-based approach is a promising tool for predicting accurate spinal cord dose in clinical practice.

Spinal tumors are neoplasm located at spinal cords, and most of them are metastases from primary cancers elsewhere. The compression of spinal tumors causes patients to undergo severe pain, and radiation therapy is a primary procedure for pain relief and tumor control. Spinal cord is a sensitive serial organ that the maximum dose to the structure is a key index of plan quality and prescription dose. Therefore, prior knowledge is important to design high-quality SBRT treatment plans. It is also noted that in contrast to radiation therapy on lung and bladder in which parts of organ can be scarified to ensure tumors receiving maximum dosage, spinal cords are a sensitive serial organ that must be protected because they control the nerve system.

There can be quantitative correlations between geometrical features and the achievable dose sparing in a number of OARs. Organ volumes and distance-to-target histogram can be modeled to estimate dose volume histogram (DVH) with the help of feature selection methods such as principal component analysis, and have given successful applications to dose planning in cancer sites such as prostate and head neck. Also, there exists experience-based mathematic formulations that can describe the relationship between the achievable mean dose and organ volumes. Image retrieval is another approach for dose prediction assuming that two patients have analogous dose distributions if they share the similar anatomical features. Also an overlap volume histogram can be provided as the spatial configuration to search for the existing patient in a database that was similar to the new patient. The dose distribution of the retrieved patient can be correspondingly assigned to the new patient.

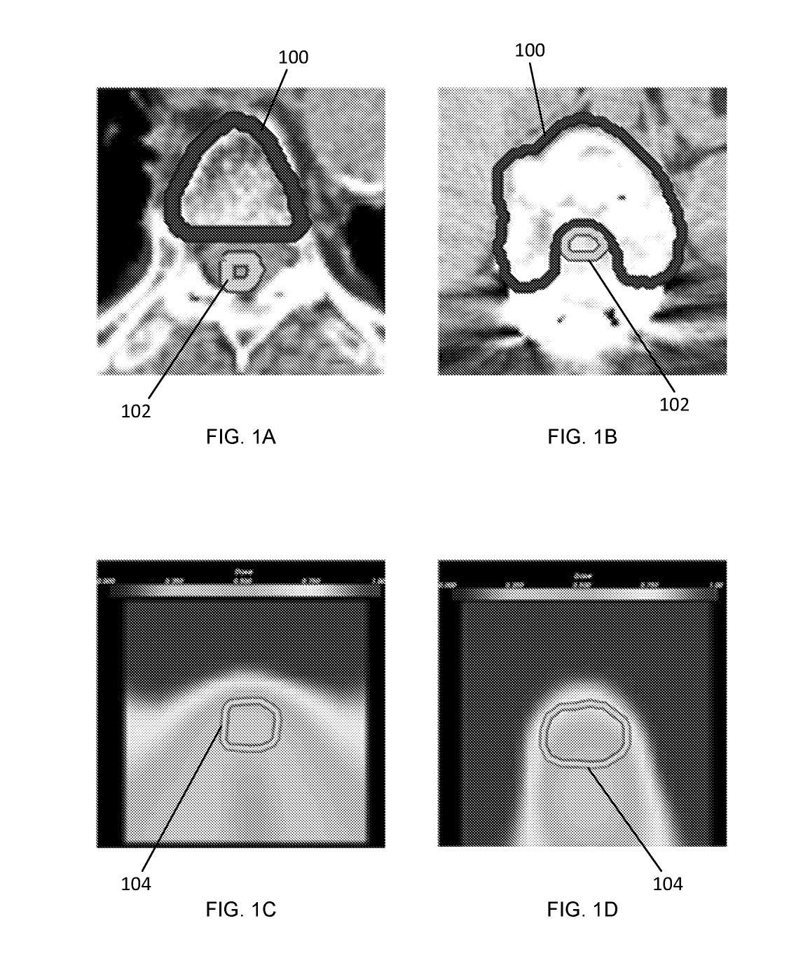

For SBRT of spine, PTV shape can be a dominant factor that affects dose levels. High dose levels at spinal cords are a typical example, represented as the PTV enclosure around the cord as shown in FIGS. 1A-1D, which are images depicting the effects of tumor enclosure on dose distributions. FIGS. 1A and 1B show spinal tumors 100 and cords 102. Dose values within curve 104 in FIG. 1D are higher than FIG. 1C, because the PTV surrounds the cord more in FIG. 1B in comparison with FIG. 1A. One way to predict dose is therefore to estimate correlation between dose distributions and PTV contour shapes. Statistical shape analysis can serve this purpose. In an example, the principal component analysis (PCA) can be applied to a set of landmarks in face images. Active shape model (ASM) was constructed to guide face recognition. Intensity values and landmark points has been embedded into the PCA, which yielded the active appearance model (AAM). However, both ASM and AAM required extensive labor work to manually select landmark points. Control points of image deformation fields can be directly imported into the PCA analysis to avoid manual labeling, and the resulting statistical model can show great accuracy in measuring shape variance of a brain image database. This can also be extended to 3D irregular heart models to estimate cardiac motion. However, applying these methods to predict dose at spinal cords can be challenging because they focused on the statistical description of shape variance to assist organ segmentation and registration. AAM established on a set of sparse points also fails to generate accurate dose prediction in small spinal cords.

Presented herein is an active optical flow model (AOFM) for measuring shape influence of PTV contours on dose prediction in spinal cords with various contributions. For example, computed optical flow can be used to measure the spatial variations of all points at spinal cords. In another example, import dose values and optical flow vectors into the PCA analysis can be used to statistically measure dose variance at spinal cords. ASM of the PTV contours can be established to statistically represent their shape variance. Further, multiple-stepwise regression methods can be used to compute correlation between AOFM and ASM. Correlation parameters can be used to predict voxel-level dose prediction at spinal cords of new patients.

In accordance with embodiments, systems and methods disclosed herein provide a framework including data preprocessing, active optical flow modeling, active shape modeling, correlation estimation, and dose prediction. In this framework, dose distribution is treated as images with dose value as the intensity at every image point.

In an example implementation and experiment, thirty spinal SBRT plans were evenly divided into training and testing datasets (Fifteen patients each) in this study. They were 4 C-spine, 6 L-spine and 20 T-spine SBRT plans, with PTV size ranging in 13.24-982.8 cm³ (mean±std.: 116.68±175.34 cm³), and the affected cord volume range in 0.62-16.04 cm³ (mean±std.: 4.49±4.12 cm³). The prescription dose range was 14.25-25 Gy (mean±std.: 18±3 Gy) in 2-5 fractions. The dose difference was measured between the predicted and clinical dose at 2% volume in the DVH (D₂), 5% (D₅), and 10% (D₁₀), common strategies to evaluate the quality of spine SBRT plans in the clinical settings.

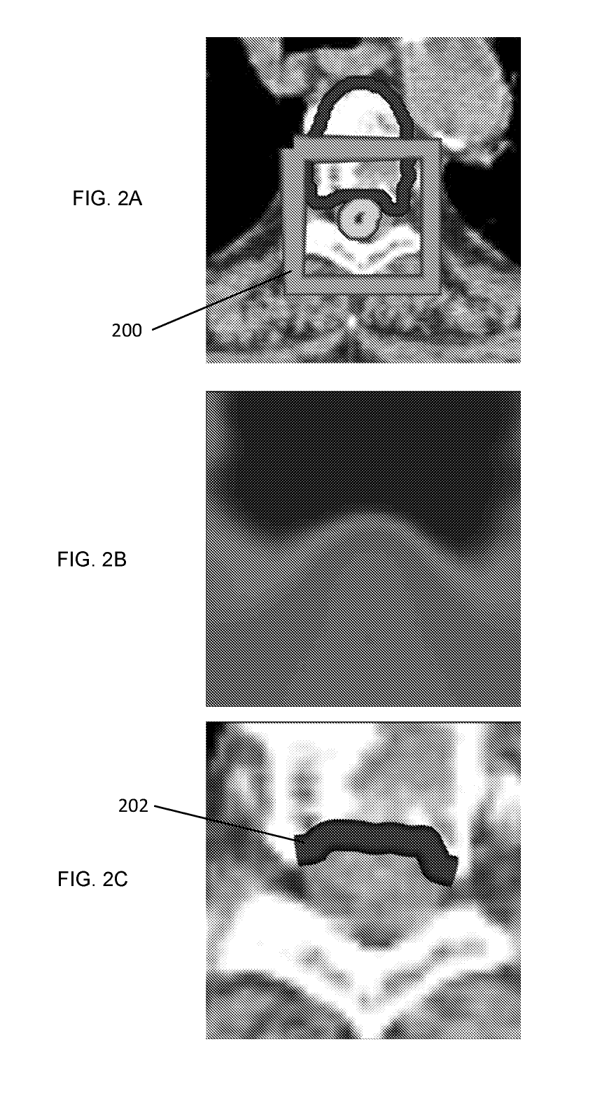

In a data preprocessing step, a sequence of dose sub-images and PTV contours adjacent to spinal cords from SBRT plans can be extracted. For example, FIGS. 2A-2C are images showing the extraction of dose sub-images and PTV contours to build AOFM and ASM. In FIG. 2A, a square 200 at the cord center shows the spatial range of the sub-image. FIG. 2B shows the dose sub-image. FIG. 2C shows a curve 202 representing the PTV contours near the spinal cord. AOFM is constructed in the sub-images because dose at spinal cords are only affected by local PTV contours.

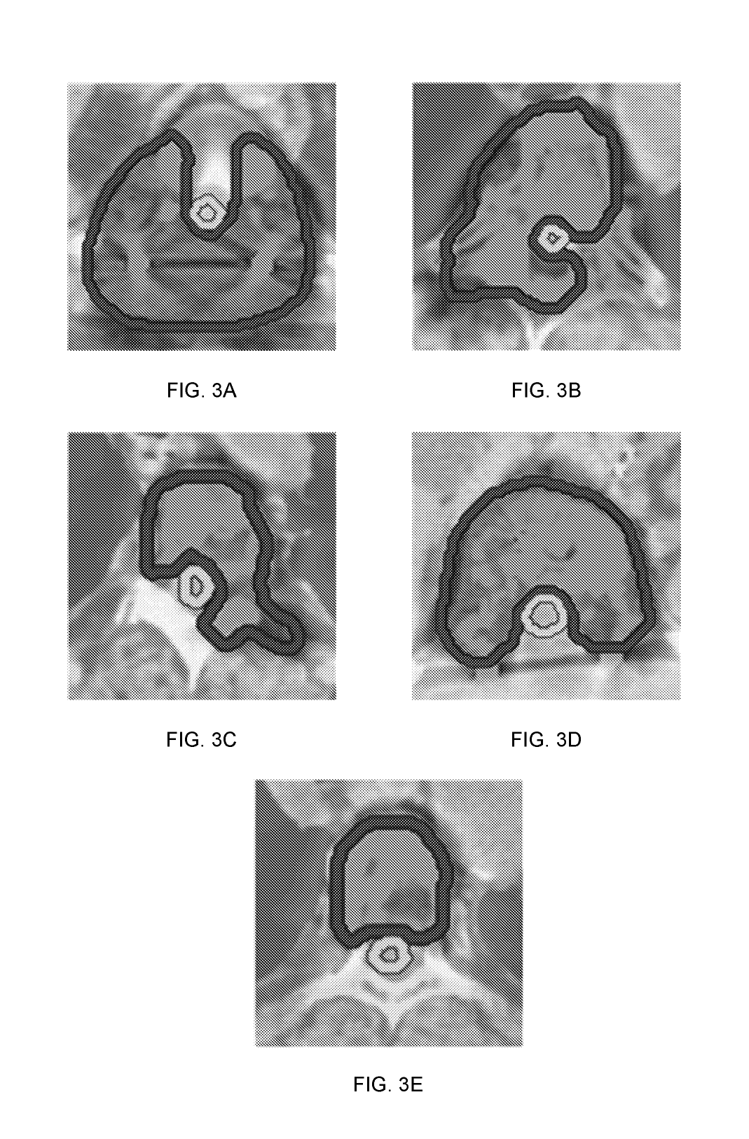

The size of the dose sub-image is 41×41 pixels indicated as the square 200 in FIG. 2A (approximately 41 mm if intra-spacing is 1.0 mm), because the diameter of spinal cord is 10-15 mm and PTV have minor effect on spinal cord if their distance is larger than 10 mm. Thus, 41 pixels are sufficient to include all types of PTVs located at different sides of the cord while still preserving the accuracy of measuring boundary effects on dose prediction. Subsequently, PTV contours were extracted that are adjacent to spinal cords as shown in FIG. 2C, because PTV and cord contours are available in the SBRT plan. Finally, all dose sub-images and PTV contours are classified into five groups in terms of spatial relationships between PTVs and cords as shown in FIGS. 3A-3E, which are images depicting five types of spatial relationships between spinal cords and PTVs, where dose images are overlaid on the CT images.

In an active optical flow model, dose sub-images are used to compute AOFM, which is essentially a statistical model to describe the dose distributions within a group. Estimating AOFM can involve optical flow computation and principal component analysis.

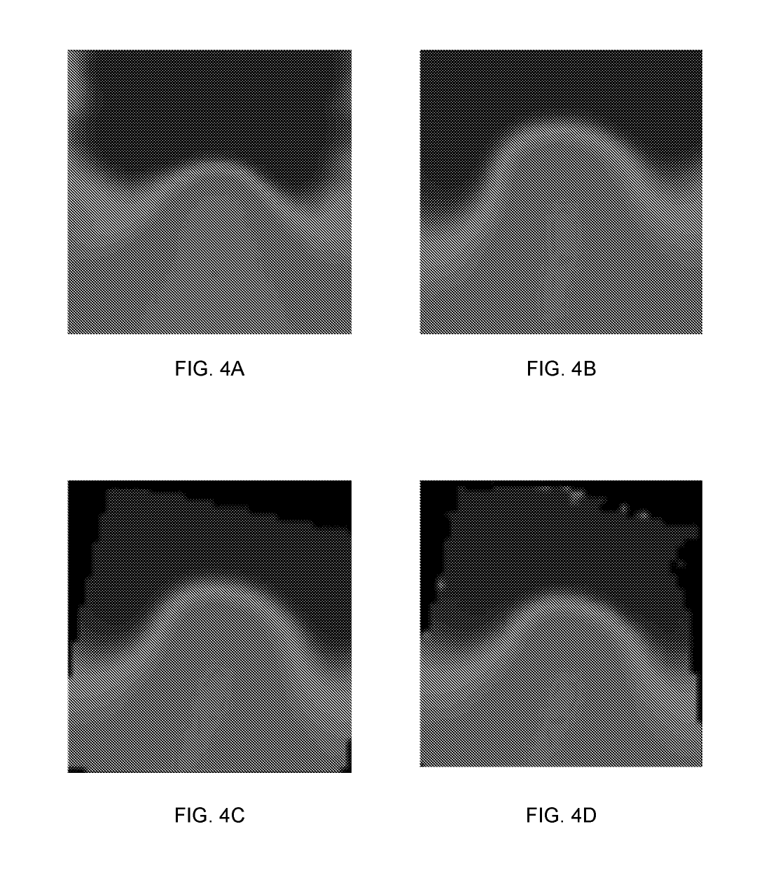

In optical flow computation, a dose image Dᵣ(x, y) in each group can be randomly chosen as the reference image. For example, FIGS. 4A-4D are images depicting an example optical flow computation. Particularly, FIG. 4A shows a reference dose image, FIG. 4B shows a current dose image, FIG. 4C shows a transformed current dose image after rigid registration, and FIG. 4D shows a transformed image after optical flow computation. Rigid image registration can be performed to remove global motion between the reference image and the current dose image (see FIG. 4B), and to generate the registered image D_{g}(X, y) (see FIG. 4C). Because D_{g}(x,y) is transformed to the reference image coordinate, optical flow computation becomes meaningful to measure local deformation between the registered image and the reference image. Let u=(uₓ, u_{y}) be the optical flow vector at a point p=(x, y) in the sub-image domain 12. The optical flow computation can be formulated as a global energy functional within a variational framework.

E(u)=∫∫(ψ(|D_{g}(p+u)−Dᵣ(p)|²)+αψ(|∇D_{g}(p+u)−∇Dᵣ(p)|²)+βψ(|∇uₓ|²+|∇u_{y}|²)) (1)

where ψ(s²)=\sqrt{s²+0.001²} is a modified L1 norm that allows for handling outliers. α and β are constant values to balance different terms. Minimizing Eq. 1 generates an optical flow field. FIG. 4D gives the result using optical flow vectors to transform FIG. 4C. Performing optical flow computation on all other images to register with the reference image yields a sequence of optical flow fields in the current group.

During principal component analysis (PCA), PCA was performed on M optical flow fields to establish AOFM, where M is the number of sub-images in the current group and each sub-image defines a feature vector x=(uₓ¹, . . . uₓ^{N}, u_{y}¹, . . . , u_{y}^{N}, d¹, . . . , d^{N}). N is the number of pixels in Ω and dⁱ is the dose value at i-th pixel. Any feature vector x can be approximated using the following equation:

x=x̅+Φ_{f}b_{f} (2)

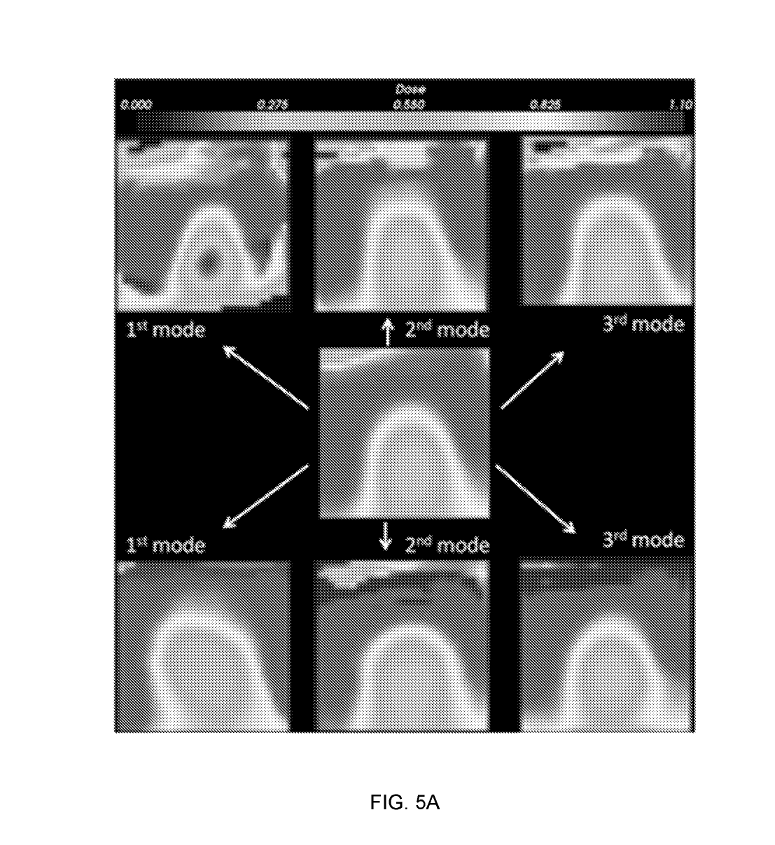

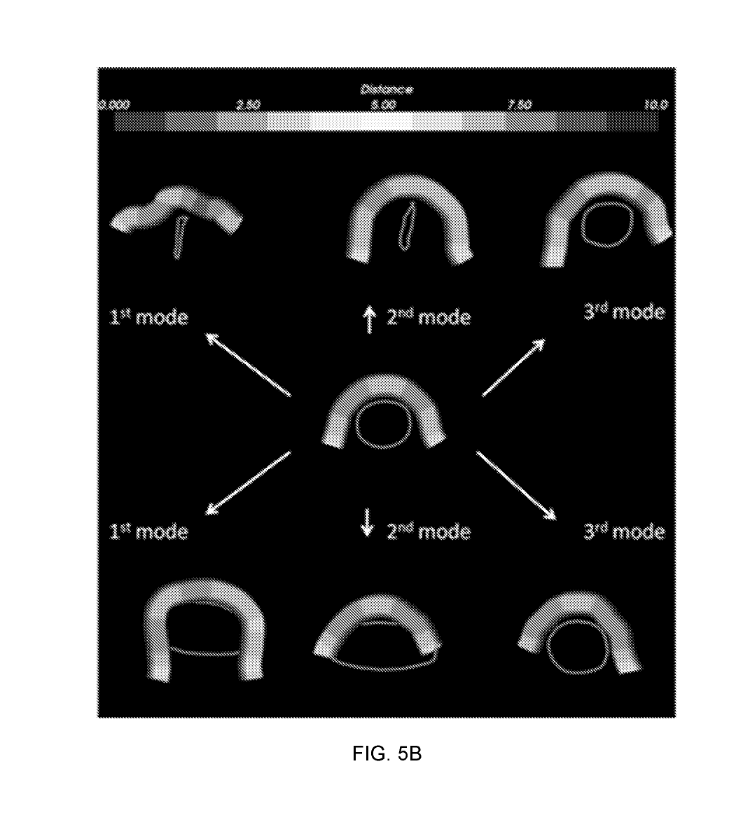

Here, x̅ is the average feature vector, Φ_{f} is formed by the eigenvectors of the covariance matrix, and b_{f} is the vector of principal component scores. FIGS. 5A and 5B are images showing instances of AOFM and ASM, respectively. Each image has been generated by varying the first three modes of variation between −3\sqrt{λᵢ} (top row) and +3\sqrt{λᵢ} (bottom row). λᵢ is the i-th eigenvalue of the AOFM or ASM. The middle row corresponds to the average mode. In the right image, the distance values between PTV contours and spinal cords (circles in FIG. 5B) are also mapped to the PTV contours and the greyscaling indicates small to large values. FIG. 5A shows the variance of AOFM corresponding to the group in FIG. 3E by setting the first parameter of b_{f} to ±3\sqrt{λ₁}, ±3\sqrt{λ₂} and ±3\sqrt{λ₃}, where λ₁≧ . . . ≧λ_{N} are eigenvalues of the covariance matrix.

Similarly, adapt ASM can be adapted to measure the shape variance of PTV contours in the current group.

y=y̅+Φₛbₛ (2)

Each feature vector y includes PTV locations, cord locations, and distance between PTV and cords. FIG. 5B shows the ASM of PTV contours. It can be observed that dose values in the cord increase in proportion to the extent of PTV enclosure on spinal cords.

In a correlation estimation, this step quantitatively measures correlation between AOFM and ASM using the multiple stepwise regression method). Principal component scores, b_{f} and bₛ of AOFM and ASM are chosen for the estimation of correlation parameters since b_{f}=(x−x̅)·Ψ_{f}^{T} and bₛ=(y−y̅)·Ψₛ^{T} can normalize the feature vectors in two models. The first 11 components of b_{f} and bₛ are empirically selected to estimate correlation parameters r satisfying b_{f}=r·bₛ.

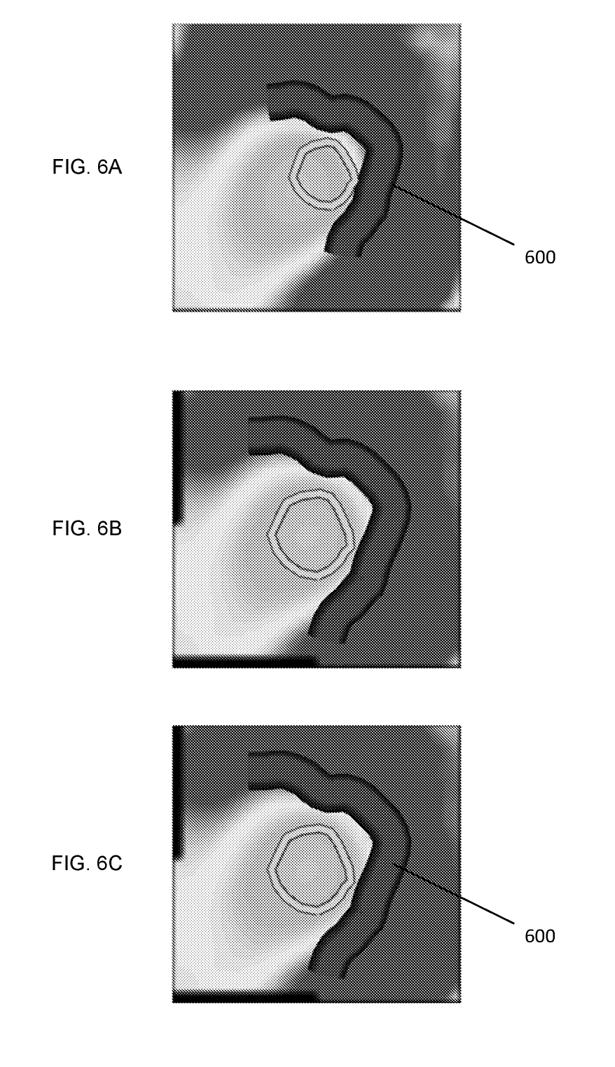

For dose prediction, a sequence of image slices containing both spinal cords and PTVs are first determined in a new CT plan. In each slice, PTV contours were extracted and a shape feature vector y was formulated. A group in FIGS. 3A-3E may subsequently be selected by searching for the shortest distance between the current PTV contour and the average contour of the group. The principal component score of the current contour is calculated as bₛ=(y−y̅)·Ψₛ^{T}, and the score of AOFM is b_{f}=r·bₛ. The feature vector containing dose values and optical flow vectors can be derived as x=r·bₛ·Ψ_{f}^{T}+x̅. FIGS. 6A-6C are images depicting a process of dose prediction. Particularly, FIG. 6A shows an initial dose in the reference image coordinate, FIG. 6B shows a final dose in the patient coordinate, and (c) clinical dose.

The initial dose shown in FIG. 6A can be reconstructed from x. However, the initial dose is represented in the reference image coordinate of the selected group, and the dose image is transformed into the patient space by applying iterative closest point algorithm to match the PTV contours in the reference image and the current image (curves 600 in FIGS. 6A and 6C). FIG. 6B gives the transformed result, and as shown, it is comparable with the actual clinical plan in FIG. 6C. Finally, dose distributions at spinal cords are predicted by applying the same strategy to all other image slices that contain PTVs and spinal cords.

Table 1 below shows dose levels between prediction and clinical plans at D₂, D₅ and D₁₀ on 15 SBRT plans in the testing dataset. Pair t-test indicated that prediction and clinical plans have no significant difference, D₂ (p=0.95), D₅ (p=0.8295), and D₁₀ (p=0.9036). The D₅ measurements (marked by *) were quite different in the 13^{th} case because the PTV contours were split into two components in some image slices. Such variance can cause the model to generate inaccurate prediction.

TABLE 1
Comparison between prediction and clinical plans
at D₂, D₅, and D₁₀ on the testing dataset.

| | D₂ (Gy) | | D₅ (Gy) | | D₁₀ (Gy) | |
|---|---|---|---|---|---|---|
| Index | Clinical | Prediction | Clinical | Prediction | Clinical | Prediction |
| 1 | 10.1 | 11.6 | 9.3 | 10.7 | 8.6 | 9.7 |
| 2 | 12.1 | 11.7 | 11.8 | 11.4 | 11.3 | 11.1 |
| 3 | 14.1 | 13.7 | 13.1 | 12.5 | 12.1 | 11.7 |
| 4 | 8.9 | 9.0 | 8.3 | 8.3 | 7.7 | 7.7 |
| 5 | 10.7 | 9.6 | 9.6 | 9.0 | 8.7 | 8.3 |
| 6 | 9.9 | 10.5 | 9.4 | 10.0 | 9.0 | 9.5 |
| 7 | 10.7 | 10.7 | 10.0 | 10.0 | 9.3 | 9.6 |
| 8 | 14.0 | 14.1 | 12.6 | 12.8 | 11.6 | 11.9 |
| 9 | 10.8 | 11.3 | 10.2 | 10.1 | 9.6 | 8.5 |
| 10 | 14.1 | 14.8 | 13.0 | 13.1 | 11.9 | 12.0 |
| 11 | 11.9 | 11.7 | 10.6 | 11.0 | 9.5 | 10.3 |
| 12 | 10.3 | 10.1 | 9.9 | 9.1 | 9.5 | 8.3 |
| 13 | 12.4 | 12.2 | 11.7* | 14.5* | 11.1 | 10.7 |
| 14 | 14.3 | 13.9 | 13.9 | 13.6 | 13.4 | 13.3 |
| 15 | 11.5 | 11.5 | 10.3 | 9.7 | 9.0 | 8.6 |
| Mean ± std. | 11.7 ± 1.7 | 11.8 ± 1.7 | 10.9 ± 1.7 | 11.1 ± 1.9 | 10.2 ± 1.6 | 10.1 ± 1.7 |

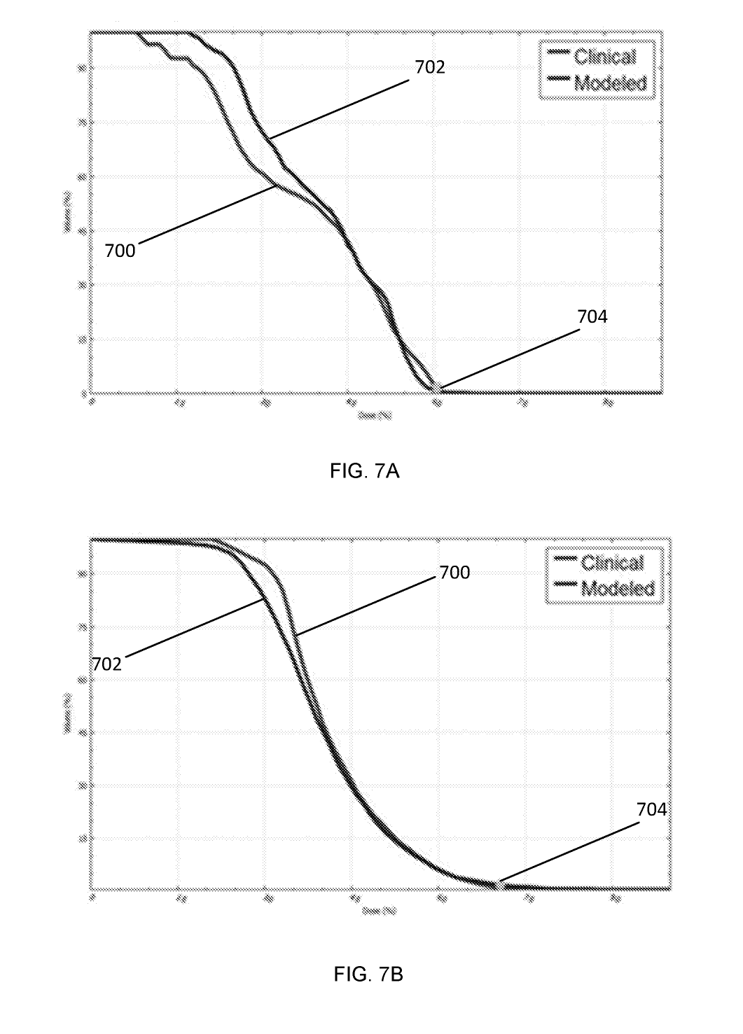

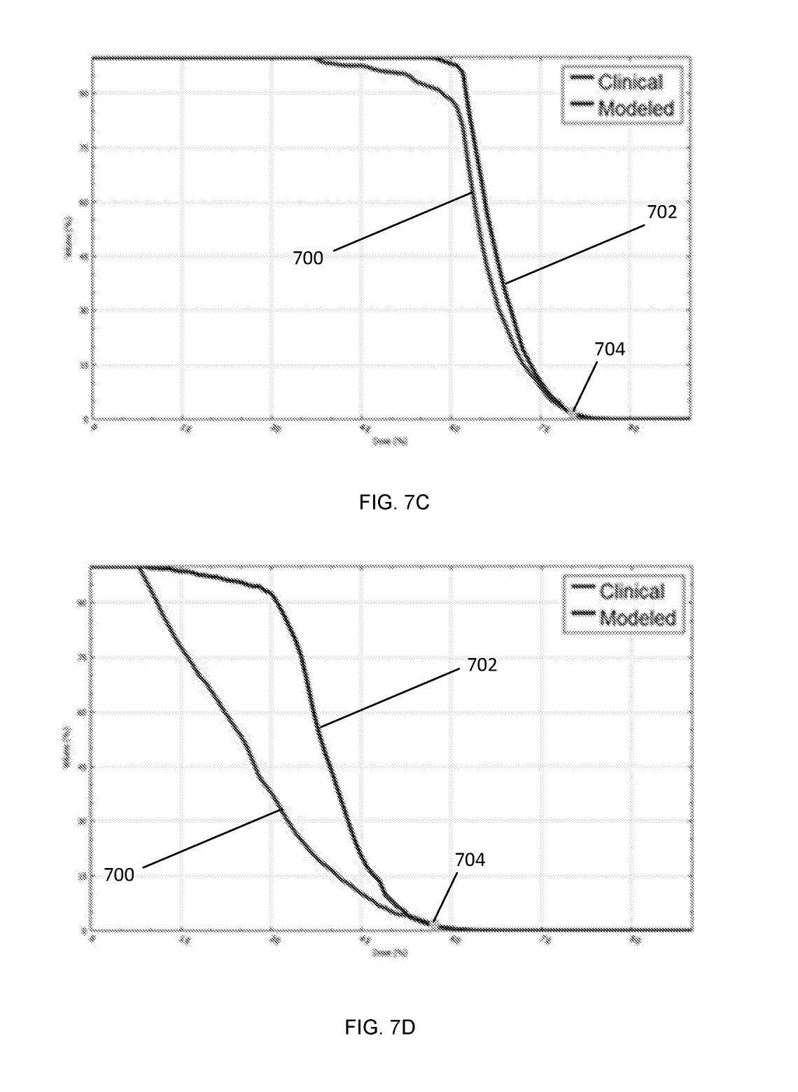

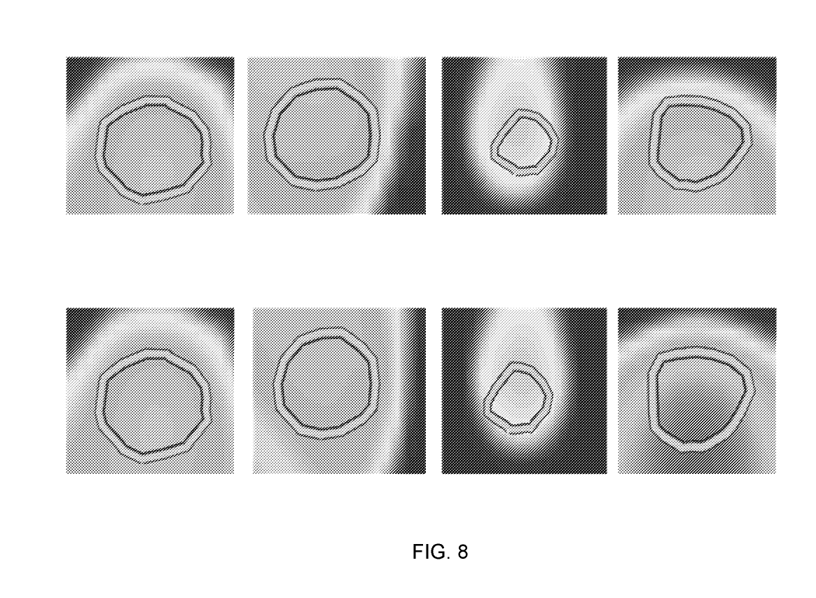

FIGS. 7A-7D are graphs showing experimental DVH results on L-spine (FIG. 7A), C-spine (FIG. 7B), and T-spine (FIGS. 7C and 7D) SBRT plans in the testing dataset. One can find that the estimated DVH (line 702) and clinical DVH (line 700) are very similar except in the low dose regions in FIG. 7D. In this case, the dose in the spinal cord is much lower than all samples in the training dataset. However, the clinical and predicated dose values at point 704 of 2% cord volume are still comparable. This value in the entire testing dataset was computed, and at the point of 2% cord volumes the dose difference between prediction and clinical plan is 3.3±3.5%.

FIG. 8 are images depicting a comparison of the predicted dose (top row) and clinical dose (bottom row). Each column corresponds to a SBRT plan in FIGS. 7A-7D. Spinal cords are represented as the curves in the center of each image. In the left column, tumors are located at the top of the spinal cord, and AOFM can predict dose very well in comparison with the clinical dose. The second column illustrated a tumor in the left side, and AOFM still predicted it very well. Similar results were observed in the third column where tumors wrapped around spinal cords from the bottom. The fourth column gave an example that our algorithm over-predicted the dose values because the training dataset failed to contain this extreme case. Thus, in FIG. 7D, the predicted DVH is higher than the clinical DVH in the low dose region. All these experimental results supported our findings in FIGS. 7A-7D.

In accordance with embodiments, disclosed herein is an active optical flow model to represent dose distributions and active shape model to measure the shape variance of tumor contours near the spinal cords. Optical flow was chosen to establish statistical model because it can accurately measure image deformation of all points between two dose images, which fulfills the purpose of accurate dose prediction in the small volume of spinal cord. The correlation parameters between AOFM and ASM were estimated via the linear regression model, and they were used to predict dose distributions in the new case. The experiments demonstrated that our algorithm accurately predicted dose with only 3% difference from the clinical dose at the point of 2% volume in the DVH graph. Since high dose level is accurate using AOFM prediction, our method can provide useful information to guide spinal SBRT planning. In the future, more training datasets will make this approach more robust.

In an experiment, the feasibility of progressive knowledge modeling for IMRT/VMAT treatment planning for multiple cancer types in pelvic region was investigated. The treatment planning knowledge model describes the quantitative correlations between patient pelvic anatomical features and the OAR DVH sparing. The model is trained by prior clinical pelvic IMRT plans using a stepwise regression machine learning technique. In an example, the progressive modeling process started with 20 low risk prostate plans (type1) which offer simplest PTV-OAR geometry. Cases with more complex PTV-OAR anatomies (prostate with lymph node, or type 2 and anal rectal, or type 3) were added to the training dataset one by one until the model prediction accuracies reach a plateau and a tentative model is saved at each step. The DVH predicted by the knowledge model for bladder, femoral heads and rectum were validated by 20, 9, and 18 cases from type 1, 2, and 3 geometries, respectively (rectum DVH is omitted for type 3). The mean and standard deviation of differences between the dosimetric parameters sampled from the DVHs and the corresponding actual plan values measures the prediction accuracy of the model. The minimum numbers of type 2 and 3 training cases required to obtain a multi-type model with optimal prediction accuracy were extracted. Its accuracy was also compared with the models trained by single type cases. Optimal prediction accuracy was obtained when 6 type 2 and 8 type 3 cases were added in training dataset. The determination coefficients R² for the OAR gEUD by the multi-type model and the single-type models, respectively are: Bladder: 0.48/0.47, rectum, 0.63/0.42 and femoral heads 0.72/0.74. The prediction accuracies by the multiple-type model and single-type model have no significant differences by F-test (p-value: bladder: 0.58, femoral head: 0.44, rectum: 0.97). The knowledge model to predict the OAR DVHs in the IMRT/VMAT treatment planning for multiple cancer types in pelvic region have comparable prediction accuracy as single-type models.

It is noted that automatic determination of beam angles may be a part of the presently disclosed subject matter. Such automatic determination is disclosed in a related patent application.

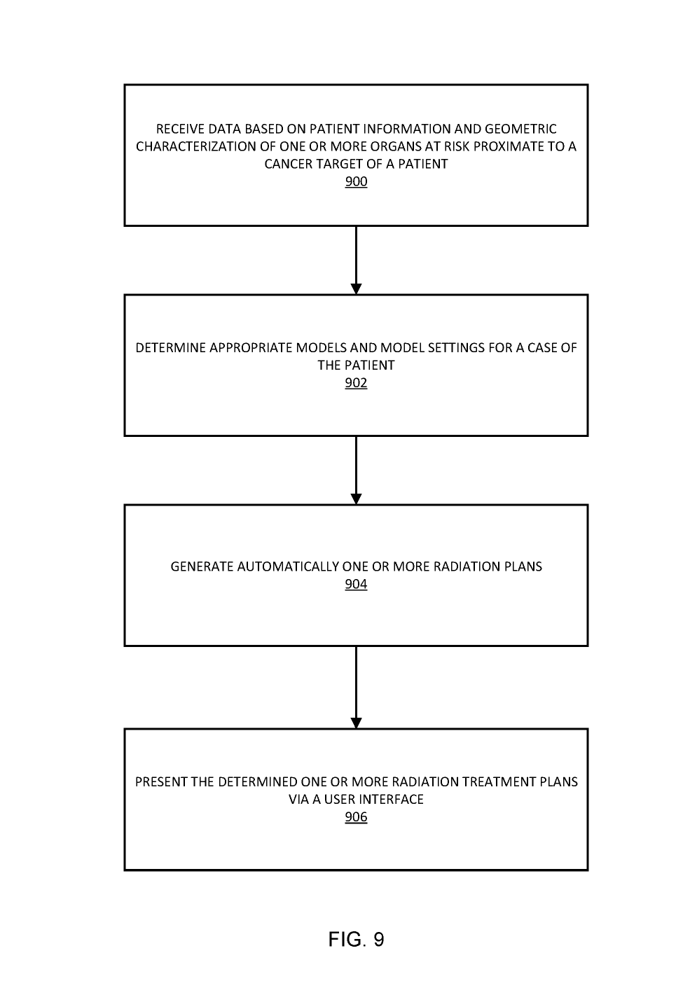

FIG. 9 illustrates a flowchart of an example method for automated radiation treatment with decision support in accordance with embodiments of the present disclosure. The method may be implemented by any suitable computing device. Example computing devices include, but are not limited to, desktop computers, laptop computers, tablet computers, smartphone, and the like. A computing device may include one or more processors and memory configured to implement the function of this example method. Further, the computing device may include one or more user interfaces such as, but not limited to, a display, a keyboard, a mouse, and the like.

Referring to FIG. 9, the method includes receiving 900 data based on patient information and geometric characterization of one or more organs at risk proximate to a target volume of a patient. For example, the patient information may include, but is not limited to, patient image, patient organ contour information, target volume contour information, clinical parameters, and the like. Further, patient information may include previous radiation treatment of the patient, previous treatment dose of the patient, location of previous radiation treatment of the patient, dose volume information of previous treatment dose of the patient, and physiological condition of the patient. In another example, patient information includes organ function analysis and/or transplant condition of the patient.

The method of FIG. 9 includes determining 902 the appropriate models and model settings for the patient. For example, one or more radiation treatment plans may be automatically generated using models learned from a plurality of radiation treatment plans of prior patient cases based on certain relationships, including one of a match or similarity, between the patient information and geometric characterization of the patient and the other patients. A radiation treatment plan may include a pattern of beam angles and dosage for use in treating a patient. Dosage information may include voxel-level dose information. In an example, a radiation treatment plan may be adjusted based on application of a beam to a critical structure such as, but not limited to, a spinal cord and/or organ at risk. The method of FIG. 9 also includes generating 904 automatically one or more radiation treatment plans using proper models learned from a plurality of radiation treatment plans of prior patient cases based on certain relationships, including one of a match or similarity, between the patient information and geometric characterization of the patient and the other patients.

The method of FIG. 9 includes presenting 906 the determined one or more radiation treatment plans via a user interface. For example, a computing device may present a radiation treatment plan via a display. As an example, a radiation treatment plan may define one or more of dose distribution and a dose volume histogram.

In accordance with embodiment, criteria for case selection is systematically learned instead of relying on manual decisions. The feasibility of using hierarchical clustering to automatically partition the set of all prior cases of clinical RT plans into subsets of cases that are suitable for modeling is investigated. First, relevant features of the prior cases that include cancer sites and patient characteristics that best categorize cases that fit into the same model are determine. Subsequently, hierarchical clustering to partition the dataset is used based on similarity of the feature vectors. Here, a suitable distance measure can be critical because the features may be both discrete and continuous. A regular simplex method may be used to convert discrete features into continuous features and use a generalized Mahalanobis distance function. To determine the suitable number of clusters, two approaches may be used. One approach may be based solely on case features and well defined cancer types. The concept of silhouette may be used to choose the number of clusters that produces the best balance between within cluster variance and cross cluster distances. The second approach can be based on the concept of regression tree. Here, the objective is to find clusters and determine models for each cluster so that the overall dose prediction error over significant cancer types is minimized.

The result of clustering is a group of case sets. Each case set can be called a modeling category and will be used to train and validate a dose prediction model. Of course, sufficient number of cases is needed to train a model. The Cook's distance may be used to make this decision. In addition to object measures of quality, clear-cut cases may be used to help ensure the sensibility of partitioning.

For each modeling category determined as described herein, the investigations may be extended into how the anatomical features G_{f}, the prescriptions D_{X}, and machine parameters M affect the dosimetric features D_{f}: D_{f}=g(D_{X},G_{f},M), where dose prescriptions D_{X} may include a set for multiple PTVs as well as clinical and biological constraints. In some studies, an array of anatomical features were investigated. Some have been explored in previous clinical studies that are associated with outcomes while others are selected based on our direct clinical experience, that is, G_{f} includes volumetric features and spatial relationships between the PTV and the OARs.

As more training cases become available, it will become feasible to develop enhanced characterization of anatomy and plan, and investigate additional features, including clinical and biological constraints and machine characteristics, that can improve the accuracy of modeling, especially for complex cases. The volumetric information has been core anatomical features in many of the clinical studies that quantify treatment toxicity, and in clinics as a direct measure of sparing efforts (DVH). Thus volumes describing doses, such as the OAR/PTV volumes, describing overlapping and beam configurations, such as the fraction of OAR volume overlapping with PTV or with the primary treatment fields, may be selected. The spatial relationship between the OARs and PTVs also affect the dose deposited in the OARs. The distance-to-target histogram (DTH) have been applied to encode such relationships. In the Euclidean space, the value of DTH at a distance bin d is the fraction of OAR volume with its maximum distance to the PTV surface less than d. Non-Euclidean distance formulas have been explored to reflect the complex IMRT dose falloffs. We will use distance formulas that capture multiple PTVs and multiple involved OARs. Features that may be added include machine characteristics M and prescription parameters D_{X}, such as prescription options, cancer stages, and institution templates.

We have experimented with support vector regression (SVR), supervised neural networks (ANN), and stepwise non-linear multivariate regression (MR). While MR showed better overall fitting, SVR seemed to capture specific plan features slightly better. The MR models that we have developed have the general form: D_{f}=Σᵢβᵢxᵢ+ε_{f}, where the predictor xᵢ represents any of the planning related features, βᵢ is the coefficient and ε_{f} is the data noise term. If ε_{f} has a zero-mean Gaussian distribution, the above formula will be a standard multiple regression model. However, the sources of the data noise not only include random statistical variation, it may also come from the systematic errors caused by outlier cases, or patient features not included in the predictors. The nature of data noise can be studied in order to improve the efficiency and accuracy of the model learning process. In the kernel based support vector regression (SVR) method, the function can be written as: D_{f}=Σ₁α₁k(w₁, \vec{x})+b, where \vec{x} is the vector of all predictors and the kernel is usually Gaussian.

With the introduction of additional features, conventional MR models will no longer suffice because the input features will include both continuous and discrete-valued variables. And compared to the number of cases available, the non-linearity of the problem also poses challenge for simple global models. A number of machine learning approaches can be used to take into account discrete-valued features: logistic regression, regression tree plus non-linear regression, regression tree plus SVR, and artificial neural network.

While beam configuration can usually be fixed for sites like prostate and HN, customized selection of beam angles is a critical component of planning for complex cases in the thorax and abdomen, as it offers another dimension to negotiate organ sparing. In current clinical practice, beam angles are often selected based on a planner's experience and adjusted through a trial-and-error process.

Translating the set of knowledge models into clinical practice is not a simple matter because clinical practice is complex and varies significantly with different patient characteristics. Given a new patient case, the following may be decided (1) which model is most suitable; and what if there is not a suitable model and there is deficiency in existing models; (2) how do we facilitate a physician's trade-off considerations; (3) what other clinical evidence, such as guidelines, policies, and templates, is relevant; and (4) how the models can be improved by the present case. In following sections we propose methods to address each of these questions.

A core component of the proposed rapid learning framework is a case-based reasoning system supported by a rules engine. As a powerful machine learning approach, case-based reasoning mimics clinical decision-making by remembering prototypical cases and prior decisions for these cases. It is a continual learning system that generally follows 4 steps: retrieve, reuse, revise, and retain. Each modeling category can be represented as a case in the system. The dose prediction model for this category and all relevant knowledge (e.g. clinical guidelines or even an automatic planning algorithm) about this category will be stored as a part of the case. For a given patient case, relevant patient features can be used to retrieve one or more prior cases, which are clusters that are most similar according to the distance function defined herein. The models and other guidance or algorithms for these cases can be applied and possibly revised to further improve the performance for the new case. A final decision can be made that is considered the best treatment for the patient. And then the new case together with the decision and reason for revision are retained to enable continuous learning, which focuses on the incremental learning of the knowledge models to be described herein. It is likely that there may be many unusual or rare planning scenarios that do not have prediction models or do not fit any of the known cases. For planning scenarios without models, we will create special cases based on analysis described herein. For these special cases, instead of models, it is the actual instances of anonymized planning data that are stored. If a new patient case matches closely to one of these special cases, we will retrieve and present the special case along with the prior clinical plans as a reference for planning. When the planner completes the planning, this new patient can be added as an additional instance of the special case. When sufficient number of instances has accumulated, the system will trigger training of a predictive model and turn the special case into a normal one. If a new patient case doesn't match any case in the system, this case can be added as a special case along with all anonymized plan related data.

Case-based reasoning has been used in a number of clinical decision support systems with several applied to radiation therapy and planning. The use of case-based reasoning is uniquely different from previous studies in that, instead of directly predicting plans or dose parameters, it may be used to select the proper dose prediction models, which will in turn predict dose parameters. Due to the severe non-linearity, the non-linear models are more suitable for predicting best-achievable dose parameters for specific patients.

Rules are often combined with case-based reasoning to support clearly defined decisions. For example, rules described herein may be used to handle categories without models, deficiencies in the models, and trigger various tradeoff scenarios to be generated. Rules may also be used to individualize the clinical guidelines and clinical trials evidence represented in ontological models. This unique ability to specialize clinical guidance to each patient regarding dose volume effects of the predicted dose levels for various organs at risk and under various tradeoff scenarios has never been reported before. The Semantic Web Rule Language (SWRL) can be used to encode integration rules.

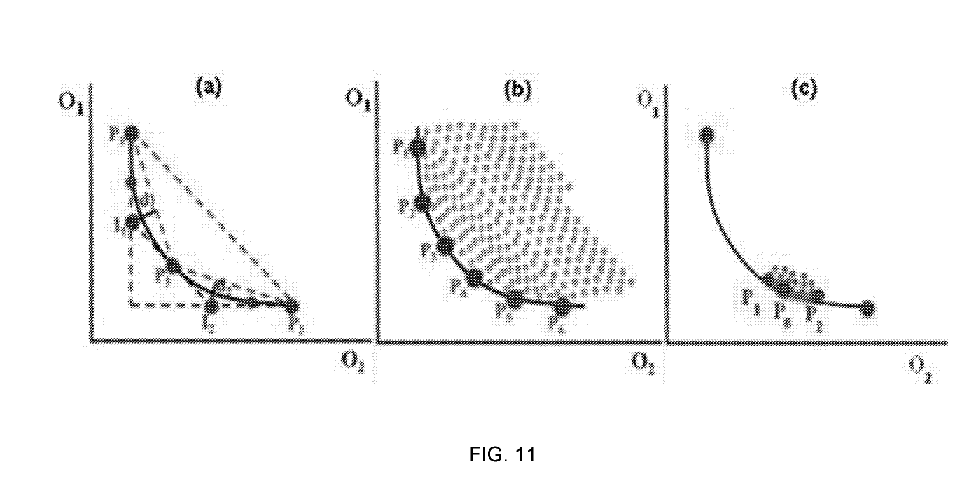

Radiation therapy planning involves the balance of multiple dosimetric objectives. The clinical tradeoff considerations between PTVs and OARs and among multiple OARs are important components of the physician's dose prescription. In general the dose distribution in one OAR depends on the prescription for the PTVs and other OARs. We have modeled the tradeoffs between two parotids in HN [14] and between PTV and OAR in spine SBRT. FIG. 11 depicts graphs of MCO engines for the Pareto front (PS) search. The solid lines are PS hence the dots on the solid lines are MCO optimal plans with different tradeoff balances. Conventional MCO engines via a) convex hulling, and (b) exhaustive search; (c) Local-MCO, where P₀ is predicted from knowledge models and P₁ and P₂ are the case specific anchor points. The local-MCO only searches the plans within the anchor points hence the final PS (green curve connecting P1 and P2) is only a portion of the conventional PS (black curve).

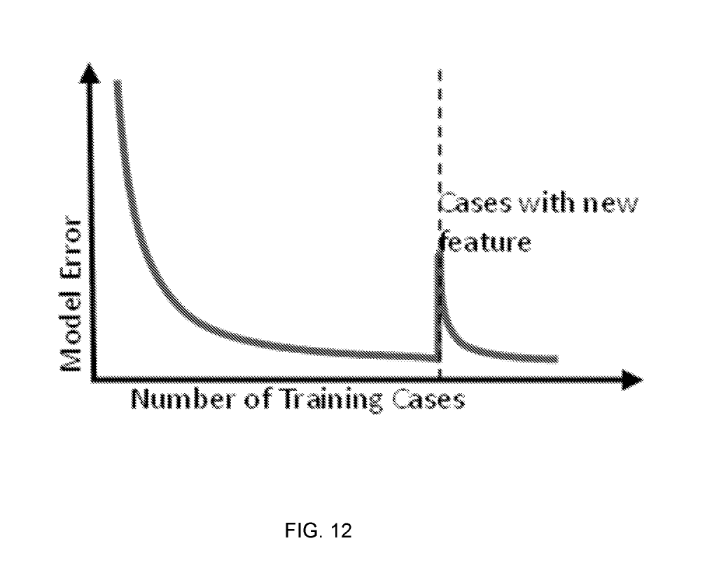

Specifically, a novel concept called local-MCO is proposed to enable trade-off considerations. A multi-criteria optimization (MCO) is often applied to optimize more than one objective simultaneously. Pareto optimization is a common strategy to search for a MCO solution in which none of the objectives can be improved without degrading the other objectives. The set of plans satisfying Pareto optimization composes Pareto fronts or Pareto surface (PS). Methods have been developed to search for Pareto optimal RT plans by either varying the optimization objectives or the optimization priorities. FIG. 12 illustrates a graph of how each MCO engine searches for the Pareto optimal plans.

The local-MCO combines knowledge models with a MCO engine so that physicians and planners not only have access to knowledge-based models, but also have the option to explore the local Pareto Surface (PS) for fine-tuning of the tradeoffs that are explicitly tailored to the specific patient's need. In the framework disclosed herein, the MCO engine does not need to search for the entire multi-objective space. Instead its search will center on the predictions given by the knowledge models and explore the Pareto front in the vicinity.

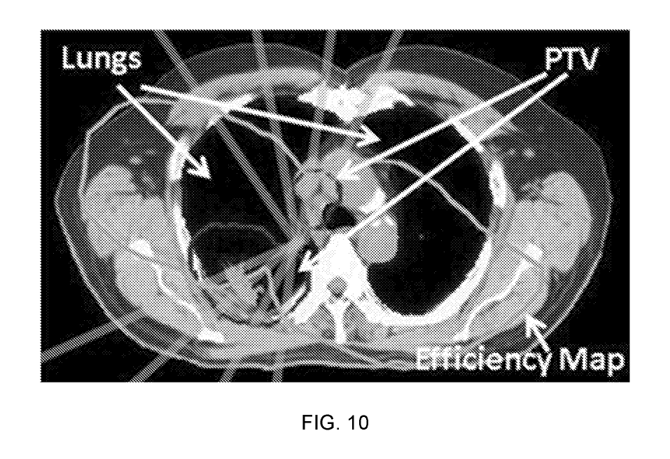

The local-MCO engine is developed and tested in the following main steps (refer to (c) of FIG. 10):

Knowledge models predict the patient specific dose optimization objectives based on the individual patient features. The knowledge models not only provide a prediction of the mean of the achievable OAR dose sparing and PTV dose coverage, it also predicts the ranges of these dosimetric parameters. The 2σ lower bounds (about 95% confidence level) of these parameters was used to determine the upper optimization objectives (these are the OAR sparing objectives) and use the upper bounds to determine the lower optimization objectives (these are the PTV coverage objectives).

Generation of a set of treatment plans near the predictions. Patient specific anchor points (P₁ and P₂ in (c) of FIG. 10) are determined when only one objective is optimized. Plans between the anchor points are generated via grid search or the convex approximation.

Extraction of Pareto surface. Non-dominant plans are first selected from the set of sample plans. These non-dominant plans are at the lower boundary of the set of plans. Then the PS is extracted by interpolation between the non-dominant plans on the objective function space.

A critical step of a rapid learning system is to build a continuous learning loop that enables evolution of knowledge models when new clinical data are collected during daily clinical practice. Such learning process mimics human knowledge accumulation, and in clinical practice, it ensures the patient treatment reflects the latest knowledge of the field. In this process, a base knowledge model is first established from an existing database. Then each subsequent case is used to continuously evaluate and update the knowledge model.

In order to decide how to update the knowledge model with a new case, a number of evaluation criteria are calculated. The studentized residual tᵢ, its t-test statistics, its leverage values hᵢᵢ, and the Cook's distance Dᵢ may be used. The studentized residual quantifies the deviation of the new case i from the current model:

$${t}_{i} = \frac{{e}_{i}}{{s ( 1 - {h}_{ii} )}^{1 / 2}}$$

where eᵢ is the residue of the prediction by the current model for the new case, s is the mean square error of the model. Leverage hᵢᵢ is the diagonal elements of the hat matrix H=X(X′ X)⁻¹X′. X is the matrix of the predictors. The leverage measures the distance of the new case in the feature space to the distribution of the other cases. Cook's distance indicates how much the model changes if the new case is included in training:

$${D}_{i} = {( \frac{{e}_{i}}{{s ( 1 - {h}_{ii} )}^{\frac{1}{2}}} )}^{2} ( \frac{{h}_{ii}}{1 - {h}_{ii}} ) \frac{1}{p}$$

where p is the number of observables. Different learning actions are triggered by these parameters.

When tᵢ is within an significance level 100(1−α)%, e.g., α=0.05, the new case is not an outlier. The learning action is to include new case and to update the current model. Different methods to train the model are investigated, e.g., step-wise multiple regression, support vector regression and kernel method.

If the new case is an outlier, the leverage hᵢᵢ is used to differentiate if the large prediction residue is due to plan quality/clinical condition variation or the new case is an isolated case in the feature space. Cases belonging to the former categories will trigger an investigation to determine whether this indicates a quality or clinical condition abnormality or this indicates a new class of cases. It may be necessary to retrain a new model for a new class of cases. On the other hand, abnormal cases or new cases in an isolated region of the feature space may trigger a re-partition of cases and retraining of all affected models.

The efficiency and accuracy are the two major evaluation endpoints for an incremental learning process. These two parameters can be quantified by the learning curve. Also note the initial decrease of model accuracy and the recovery when new features added during model training. This curve describes the longitudinal improvement of model accuracies with increasing number of training cases. In order to reduce the effect of cross-sectional data variation to the learning curve, the modeling accuracies are evaluated with a repeated random splitting cross validation method. The Prediction Sum of Squares (PRESS) of their differences and the Median Absolute Differences (MAD) are calculated for the validation cases as:

$$PRESS = {\sum }_{i = 1}^{n} {( {Y}_{i} - \hat{{Y}_{i}} )}^{2} = {\sum }_{i = 1}^{n} {e}_{i}^{2}$$

$$MAD = median \{ \vert {e}_{i} \vert \}$$

where Yᵢ and Ŷᵢ are the actual and the model predicted dosimetric parameters, respectively.

As more training samples are used, the models in their static form will reach a plateau of learning accuracy. It is believed that the complexity of models and the sophistication of learning strategies in a rapid learning framework should continue to evolve to achieve new plateaus with better optimums as more data and knowledge become available and as new RT technologies continue to advance. For both learning and evolving models, knowledge modeling should be treated as a dynamic process. As more training cases are available, more number and types of features should be incorporated, including geometrical, biological, clinical factors, and even outcomes data into the models to improve accuracy.

The knowledge models predict best achievable dose for each patient based on prior clinical data. However, the prediction only provides guidance. The optimal plan for an individual patient may require further investigation and adjustments based on patient's unique clinical conditions. To do this, physicians may need published evidence about dose volume effects of tumor and normal organs. The most recent guidelines for normal tissue effects are summarized in a set of papers in Quantitative Analysis of Normal Tissue Effects in Clinics (QUANTEC). Each organ-specific QUANTEC paper addresses the radiation effects of one or two organs and provides guidance in ten standardized categories including factors affecting risk, mathematical/biological models, recommended dose/volume limits, and Toxicity scoring. The guidelines and clinical trials results are presented in narrative formats. While the discussions are rich and insightful, the knowledge is not easily accessed or synthesized, especially at the point of care.

An ontology explicitly represents important concepts of a domain and logically describing their relationships. An ontological model allows computers to “understand” the domain knowledge and thus enables powerful logical manipulation of knowledge, including semantic query, automatic reasoning, and verification of the consistency of the knowledge model. Ontological models are important components to model clinical knowledge and provide decision support at the point of care. Especially of interest is the computerization of clinical practice guidelines knowledge that provides recommendations, rules for guidance, and automated reasoning based on patient information.

The present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

One skilled in the art will readily appreciate that the present subject matter is well adapted to carry out the objects and obtain the ends and advantages mentioned, as well as those inherent therein. The present examples along with the methods described herein are presently representative of various embodiments, are exemplary, and are not intended as limitations on the scope of the present subject matter. Changes therein and other uses will occur to those skilled in the art which are encompassed within the spirit of the present subject matter as defined by the scope of the claims.

### REFERENCES

Yuan et al., Lung IMRT Planning Using Standardized Beam Bouquet Templates, AAPM2014: SU-F-BRD-9.
Hu et al., Machine Learning Methods for Knowledge Based Treatment Planning of Prostate Cancer, AAPM2014: SU-E-T-229.
Sheng et al., Building Atlas for Automatic Prostate IMRT Planning: Anatomical Feature Parameterization and Classification, AAPM2014: MO-C-17A-7.
Lu et al., Progressive Knowledge Modeling for Pelvic IMRT/VMAT Treatment Planning, AAPM2014: TU-C-17A-11.
Liu et al., Active Optical Flow Model: Predicting Voxel-Level Dose Prediction in Spine SBRT, AAPM2014: TH-A-9A-1.
Appenzoller L M, Michalski J M, Thorstad W L, Mutic S and Moore K L 2012 Predicting dose-volume histograms for organs-at-risk in IMRT planning Med Phys. 39 7446-61
Brox T, Bruhan A, Papenberg N and Weichert J 8th European Conference on Computer Vision, (Prague, Czech Republic, 2004), vol. Series 4) ed TP a J Matas pp 25-36
Chanyavanich V, Das S K, Lee W R and Lo J Y 2011 Knowledge-based IMRT treatment planning for prostate cancer Med Phys. 38 2515-22
Cootes T F, Edwards G J and Taylor C J 2001 Active appearance models IEEE Transactions on Pattern Analysis and Machine Intelligence 23 681-5
Cootes T F, Taylor C J, Cooper D H and Graham J 1995 Active Shape Models-Their Training and Application Computer Vision and Image Understanding 61 38-59
Heimann T and Meinzer H-P 2009 Statistical shape models for 3D medical image segmentation: A review Medical Image Analysis 13 543-63
Holt T, Hoskin P, Maranzano E, Sahgal A, Schild S E, Ryu S and Loblaw A 2012 Malignant epidural spinal cord compression: the role of external beam radiotherapy Current Opinion in Supportive and Palliative Care 6 103-8
Horkaew P and Yang G Z Information Processing in Medical Imaging, 2003), vol. Series 2732) pp 13-24
Ibanez L, Schroeder W, Ng L and Cates J 2005 The ITK Software Guide: Kitware Inc.)
Kutner M, Nachtsheim C and Neter J 2004 Applied Linear Regression Models: McGraw-Hill/Irwin)
Lee K J, Barber D C and Walton L 2006 Automated gamma knife radiosurgery treatment planning with image registration, data-mining, and Nelder-Mead simplex optimization. Med Phys. 33 2532-40
Lian J, Yuan L, Ge Y, Chera B S, Yoo D P, Chang S, Yin F-F and Wu Q J 2013 Modeling the dosimetry of organ-at-risk in head and neck IMRT planning: an intertechnique and interinstitutional study Med Phys. 40 121704
Moore K L, Brame R S, Low D A and Mutic S 2011 Experience-based quality control of clinical intensity-modulated radiotherapy planning Int J Radiat Oncol Biol Phys. 81 545-51
Rueckert D, Frangi A F and Schnabel J A 2003 Automatic construction of 3-D statistical deformation models of the brain using nonrigid registration IEEE Trans Med Imaging. 22 1014-25
Sahgal A, Ma L, Gibbs I, Gerszten P C, Ryu S, Soltys S, Weinberg V, Wong S, Chang E, Fowler J and Larson D A 2010 Spinal cord tolerance for stereotactic body radiotherapy International journal of radiation oncology, biology, physics 77 548-53
Wu B, McNutt T, Zahurak M, Simari P, Pang D, Taylor R and Sanguineti G 2012 Fully Automated Simultaneous Integrated Boosted-Intensity Modulated Radiation Therapy Treatment Planning Is Feasible for Head-and-Neck Cancer: A Prospective Clinical Study International Journal of Radiation Oncology Biology Physics 84 647-53
Wu B, Ricchetti F, Sanguineti G, Kazhdan M, Simari P, Chuang M, Taylor R, Jacques R and McNutt T 2009 Patient geometry-driven information retrieval for IMRT treatment plan quality control Med. Phys. 36 5497-505
Yasushi H, Kataoka M, Senba T, Uwatsu K, Sugawara Y, Inoue T, Sakai S, Aono S, Takahashi T and Oda S 2009 Vertebral metastases with high risk of symptomatic malignant spinal cord compression Japanese Journal of Clinical Oncology 39 431-4
Yuan L, Ge Y, Lee W R, Yin F F, Kirkpatrick J P and Wu Q J 2012 Quantitative analysis of the factors which affect the interpatient organ-at-risk dose sparing variation in IMRT plans Med Phys. 39 6868-78
Yuan L, Wu Q J, Yin F-F, Jiang Y, Yoo D P and Ge Y 2014 Incorporating single-side sparing in models for predicting parotid dose sparing in head and neck IMRT Med. Phys. 41 021728
Zhang Z 1994 Iterative point matching for registration of free-form curves and surfaces International Journal of Computer Vision 13 119-52
Zhu X, Ge Y, Li T, ThongPhiew D, Yin F-F and Wu Q J 2011 A planning quality evaluation tool for prostate adaptive IMRT based on machine learning Med Phys. 38 719-26
Zhang, X., Li, X., Quan, E. M., and Li, Y.: A methodology for automatic intensity-modulated radiation treatment planning for lung cancer. Physics in Medicine and Biology. 56(13), 3873-3893 (2011)
Abraham C, Molinari N and Servien R 2013 Unsupervised clustering of multivariate circular data Statistics in Medicine 32 1376-82
Appenzoller L M, Michalski J M, Thorstad W L, Mutic S and Moore K L 2012 Predicting dose-volume histograms for organs-at-risk in IMRT planning Med Phys 39 7446-61
Breedveld S, Storchi P R, Voet P W and Heij men B J 2012 iCycle: Integrated, multicriterial beam angle, and profile optimization for generation of coplanar and noncoplanar IMRT plans Med Phys 39 951-63
Craft D 2007 Local beam angle optimization with linear programming and gradient search Phys Med Biol 52 N127-35
Das S, Cullip T, Tracton G, Chang S, Marks L, Anscher M and Rosenman J 2003 Beam orientation selection for intensity-modulated radiation therapy based on target equivalent uniform dose maximization Int J Radiat Oncol Biol Phys 55 215-24
Dong P, Lee P, Ruan D, Long T, Romeijn E, Low D A, Kupelian P, Abraham J, Yang Y and Sheng K 2013 4pi noncoplanar stereotactic body radiation therapy for centrally located or larger lung tumors Int J Radiat Oncol Biol Phys 86 407-13
Harris J P, Murphy J D, Hanlon A L, Le Q T, Loo B W, Jr. and Diehn M 2014 A Population-Based Comparative Effectiveness Study of Radiation Therapy Techniques in Stage III Non-Small Cell Lung Cancer Int J Radiat Oncol Biol Phys 88 872-84
Ji K, Zhao L J, Liu W S, Liu Z Y, Yuan Z Y, Pang Q S, Wang J and Wang P 2014 Simultaneous integrated boost intensity-modulated radiotherapy for treatment of locally advanced non-small-cell lung cancer: a retrospective clinical study The British journal of radiology 87 20130562
Jia X, Men C, Lou Y and Jiang S B 2011 Beam orientation optimization for intensity modulated radiation therapy using adaptive 1(2,1)-minimization Phys Med Biol 56 6205-22
Jonker R and Volgenant A 1987 A Shortest Augmenting Path Algorithm for Dense and Sparse Linear Assignment Problems Computing 38 325-40
Kaufman L and Rousseeuw P J 2009 Finding Groups in Data (Hoboken, N.J.: John Wiley & Sons, Inc.)
Li Y, Yao J and Yao D 2004 Automatic beam angle selection in IMRT planning using genetic algorithm Phys Med Biol 49 1915-32
Liao Z X, Komaki R R, Thames H D, Jr., Liu H H, Tucker S L, Mohan R, Martel M K, Wei X, Yang K, Kim E S, Blumenschein G, Hong W K and Cox J D 2010 Influence of technologic advances on outcomes in patients with unresectable, locally advanced non-small-cell lung cancer receiving concomitant chemoradiotherapy Int J Radiat Oncol Biol Phys 76 775-81
Pugachev A, Li J G, Boyer A L, Hancock S L, Le Q T, Donaldson S S and Xing L 2001 Role of beam orientation optimization in intensity-modulated radiation therapy Int J Radiat Oncol Biol Phys 50 551-60
Schreibmann E and Xing L 2004 Feasibility study of beam orientation class-solutions for prostate IMRT Medical Physics 31 2863
Shirvani S M, Juloori A, Allen P K, Komaki R, Liao Z, Gomez D, O'Reilly M, Welsh J, Papadimitrakopoulou V, Cox J D and Chang J Y 2013 Comparison of 2 common radiation therapy techniques for definitive treatment of small cell lung cancer Int J Radiat Oncol Biol Phys 87 139-47
Wang X, Zhang X, Dong L, Liu H, Gillin M, Ahamad A, Ang K and Mohan R 2005 Effectiveness of noncoplanar IMRT planning using a parallelized multiresolution beam angle optimization method for paranasal sinus carcinoma Int J Radiat Oncol Biol Phys 63 594-601
Yuan L, Ge Y, Lee W R, Yin F F, Kirkpatrick J P and Wu Q J 2012 Quantitative analysis of the factors which affect the interpatient organ-at-risk dose sparing variation in IMRT plans Med Phys 39 6868-78
Zhang H H, Gao S, Chen W, Shi L, D'Souza W D and Meyer R R 2013 A surrogate-based metaheuristic global search method for beam angle selection in radiation treatment planning Phys Med Biol 58 1933-46
Zhang X, Li X, Quan E M, Pan X and Li Y 2011 A methodology for automatic intensity-modulated radiation treatment planning for lung cancer Physics in Medicine and Biology 56 3873

What is claimed is:

1. A method comprising:
using at least one processor and memory for:
receiving data based on patient information and geometric characterization of one or more organs at risk and a cancer target of a patient;
determining appropriate models and model settings for a case of the patient;
generating automatically one or more radiation treatment plans using proper models learned from a plurality of radiation treatment plans of prior patient cases based on certain relationships, including one of a match or similarity, between the patient information and geometric characterization of the patient and the other patients; and
presenting the determined one or more radiation treatment plans via a user interface.

2. The method of claim 1, wherein the patient information includes one or more of patient image, patient organ contour information, target volume contour information, and clinical parameters.

3. The method of claim 1, wherein the models comprise voxel level models that characterize geometric and dose variations and their relationships.

4. The method of claim 3, wherein the geometric and dose variations are characterized by active optical flow model (AOFM) and active shape model (ASM).

5. The method of claim 1, wherein the plurality of radiation treatment plans are each associated with data indicating patient anatomy and cancer target,
wherein the method further comprises analyzing the data indicating patient anatomy and cancer target to generate mathematically parameterized patterns; and
wherein generating automatically one or more radiation treatment plans comprises using the models to predict the dose constraint parameters and optimization parameters and apply these parameters to plan optimization; and
wherein generating the multiple radiation treatment plans comprises using a local MCO strategy to ensure all the alternative plans are Pareto optimal.

6. The method of claim 1, wherein the plurality of radiation treatment plans each include a configuration of beam angles, and
wherein presenting the determined one or more radiation treatment plans comprises presenting information about the configuration of beam angles of the one or more radiation treatment plans.

7. The method of claim 1, wherein the plurality of radiation treatment plans each include dose distribution information, and
wherein presenting the determined one or more radiation treatment plans comprises presenting the dose distribution information of the one or more radiation treatment plans.

8. The method of claim 7, wherein the dose distribution information includes voxel-level dose information.

9. The method of claim 7, wherein determining one or more radiation treatment plans comprises adjusting the one or more radiation treatment plans based on modification of dose distribution to a critical structure.

10. The method of claim 9, wherein the critical structure comprises one of a spinal cord and other organ at risk.

11. The method of claim 1, wherein the patient information includes one of previous radiation treatment of the patient, previous treatment dose of the patient, location of previous radiation treatment of the patient, dose volume information of previous treatment dose of the patient, physiological condition of the patient, patient preference and treatment goals, and other treatment related information.

12. The method of claim 1, wherein the patient information includes one of organ function analysis and transplant condition of the patient.

13. The method of claim 1, further comprising:
receiving selection of one of the determined one or more radiation treatment plans via the user interface;
receiving input for adjusting the dose volume histogram and/or dose distribution of selected one of the determined one or more radiation treatment plans via the user interface; and
adjusting the selected one of the determined one or more radiation treatment plans based on the input.

14. The method of claim 1, wherein the geometric characterization associates each of a plurality of distances from the target volume with a respective percentage for the volume of the one or more organs at risk.

15. The method of claim 1, wherein the data comprises the size of the target volume and the respective sizes and shapes of the one or more organs at risk.

16. The method of claim 1, wherein the geometric characterization comprises measures of the extent of cancer target (PTV) wrapping around an organ at risk.

17. The method of claim 1, wherein the models comprise information about one of radiation treatment knowledge, experience, and preferences, and computerized models of published clinical trials results and guidelines.

18. The method of claim 1, wherein the radiation treatment plans define at least one of a dose distribution and a dose volume histogram.

19. The method of claim 1, wherein determining the appropriate models and model settings comprises using a case based reasoning technique.

20. The method of claim 1, wherein determining the appropriate models and model settings comprises automatically determining a set of models that best cover a plurality of prior patient cases.

21. The method of claim 20, wherein automatically determining a set of models comprises one of using clustering analysis and regressive tree.

22. The method of claim 1, wherein the plurality of radiation treatment plans are learned dynamically.

23. The method of claim 22, further comprising:
collecting data associated with radiation treatment plans; and
learning and adding to the radiation treatment plans based on the collected data.

24. The method of claim 1, wherein the models comprise voxel level models that characterize geometric and dose variations and their relationships

25. A system comprising:
at least one processor and memory configured to:
receive data based on patient information and geometric characterization of one or more organs at risk and a cancer target of a patient; and
determine appropriate models and model settings for a case of the patient;
generate automatically one or more radiation treatment plans using proper models learned from a plurality of radiation treatment plans of prior patient cases based on certain relationships, including one of a match or similarity, between the patient information and geometric characterization of the patient and the other patients; and
a user interface configured to present the determined one or more radiation treatment plans.

26. The system of claim 25, wherein the plurality of radiation treatment plans are each associated with data indicating patient anatomy,
wherein the at least one processor and memory configured to:
analyze the data indicating patient anatomy to generate mathematically parameterized patterns; and
use the patterns to match the patient with one or more of the other patients, wherein the determined radiation treatment plans are the radiation treatment plans of the matched one or more of the other patients.

27. The system of claim 25, wherein the plurality of radiation treatment plans each include a pattern of beam angles, and
wherein the user interface is configured to present information about the pattern of beam angles of the one or more radiation treatment plans.

28. The system of claim 25, wherein the plurality of radiation treatment plans each include beam dosage information, and
wherein the user interface is configured to present the beam dosage information of the one or more radiation treatment plans.

29. The system of claim 28, wherein the beam dosage information includes voxel-level dose information.

30. The system of claim 28, wherein the at least one processor and memory configured to adjust the one or more radiation treatment plans based on application of a beam dose to a critical structure.

31. The system of claim 30, wherein the critical structure comprises one of a spinal cord and organ at risk.
